(19) United States
(12) Patent Application Publication
(10) Pub. No.: US 2021/0079481 A1
(43) Pub. Date: Mar. 18, 2021

(54) DIAGNOSTIC ASSAY FOR CANCER

(71) Applicant: Stitch Bio, LLC, Waltham, MA (US)

(72) Inventor: Anthony P. Shuber, Northbridge, MA (US)

(21) Appl. No.: 17/022,248

(22) Filed: Sep. 16, 2020

Related U.S. Application Data
(60) Provisional application No. 62/901,099, filed on Sep. 16, 2019.

(51) Int. Cl. C12Q 1/6886

(52) U.S. Cl. CPC C12Q 1/6886; C12Q 2600/158

(57) ABSTRACT

The invention provides methods of detecting cancer in a subject. The methods of the invention entail monitoring accumulation of passenger fusions over time in a subject. An increase in the level of passenger fusions over time is indicative of the presence of cancer in the subject.

### FIELD OF THE INVENTION

The invention relates to molecular diagnostics.

### BACKGROUND

Cancer is a leading cause of death and a significant cost to the global health economy. Numerous efforts have focused on early detection as a means of reducing both morbidity and cost.

Cancer is generally considered to be a disease associated with genomic instability. As such, most, if not all, approaches to early detection focus on non-germline mutations and, in particular, single nucleotide polymorphisms that are associated with cancer. Recently, it has been recognized that structural variations, such as rearrangements and indels, are present in many cancers. In particular, cancer cell DNA often contains large deletions, which often result in loss of heterozygosity fusions in which large pieces of genomic DNA are deleted. A problem with cancer diagnostics is that the presence of fusions is not necessarily indicative of the presence of cancer. Somatic cells may experience genomic mutations that do not proceed to cancer. Typically, when that happens, cellular repair mechanisms, cell cycle regulators and/or the immune system will intervene to halt further division of the damaged cells. Thus, the mere presence of DNA fusions is not a reliable diagnostic for cancer and may lead to unnecessary diagnostic and therapeutic procedures.

### SUMMARY

The invention contemplates the use of fusions resulting from deletions in genomic DNA as a novel analyte to assess oncogenic risk. In particular, the invention recognizes that the velocity of changes in fusions in cancer-associated passenger mutations is highly-predictive of disease onset. “Passenger” mutations are mutations that are assumed to have no direct causative role in the etiology of cancer, as opposed to so-called “driver” mutations, which are thought to increase the net cellular proliferation typically associated with cancer. The invention recognizes that the mere presence of passenger fusions is not in itself diagnostic of disease. Rather, it is the accumulation, typically represented by the slope of change in the number of such mutations, that is diagnostically-relevant. In that regard, the velocity of change in passenger fusions is to cancer generally as rising PSA (prostate-specific antigen) is to prostate cancer specifically. Like, PSA, the detection of passenger fusions alone is insufficient for sensitive and specific diagnosis of cancer. Accordingly, the invention provides significant improvements, especially in the specificity of cancer diagnostics, as the invention reduces the necessity for invasive diagnostic testing or therapeutic measures which ultimately may not be necessary. It is the accumulation or velocity of fusions in passenger mutations that is the most accurate indicator of the presence of disease.

Accordingly, the invention provides methods of detecting cancer by monitoring the accumulation of passenger fusions over time. Thus, methods of the invention include using passenger fusions as an analyte in an assay to detect cancer. In methods of the invention, passenger fusions may be assessed in bulk to determine generally whether the quantity of mutations is increasing or may themselves be quantified, preferably on a weighted basis with respect to a standard, over time. In one embodiment patient samples are obtained from both germline sources (e.g., buccal cells) and from blood or tumor tissue. Passenger mutations are identified by comparing DNA obtained from blood or tumor tissue to germline DNA. If fusions in passenger genes (passenger mutations) are detected, the analysis is repeated at future time points in order to determine a rate of change of the passenger fusions. The rate of change may be quantitative or based on bulk measurement. For example the slope of a curve defining a rate of passenger fusion accumulation defines the acceleration of passenger fusion accumulation, which is then assessed to provide an informative diagnosis.

Methods of the invention include quantifying passenger fusions in a sample obtained from a subject at an initial time, or first point in time, and at a subsequent time, wherein an increase in the number or amount of such mutations over time is indicative of cancer. For example, a passenger fusion may be identified in a subject by comparing a whole-genome sequence of germline DNA of the subject to DNA obtained from blood or tissue to identify passenger fusions in the latter. The germline DNA may be obtained from any type of biological sample of the subject that contains germline DNA. Preferably, the germline DNA is obtained from buccal cells. The test DNA may be obtained from a suspected tumor or from blood, including plasma and serum. Any passenger fusions identified in test samples are specific to the subject, and thus are a patient-specific biomarker that is used to monitor the subject.

Methods of the invention include obtaining a baseline level of the passenger fusion in an initial test sample obtained from the subject. The baseline level, or the initial passenger fusion index, may be obtained by quantifying any passenger fusions identified in the sample by comparison to the germline DNA of the same patient. The initial passenger fusion index may be used to compare to the levels of the passenger fusion in subsequent samples obtained from the same patient.

Methods of the invention include quantifying passenger fusions in a subsequent sample obtained from the subject at a later time to determine whether there is an increase in the level of the passenger fusions. Subsequent samples may be obtained at any future time point from the subject. For example, a subsequent sample may be obtained monthly, yearly, or at any frequency. Identifying an increase in the level of passenger mutations in subsequent samples may indicate the presence of cancer in the subject. The frequency of obtaining subsequent samples may be increased if an increase in the level of the passenger fusion is identified at any time point. For example, subsequent samples may be obtained on a monthly basis to monitor the increasing level of the passenger fusions. The frequency of the subsequent samples may be decreased if a decrease in the level of the passenger fusion is identified. Each time, comparing the level of passenger fusions in the subsequent sample to the baseline level, thereby monitoring for an increase in the level of passenger fusions. Alternatively, the level of passenger fusions of the subsequent sample may be compared to a previous sample. Regardless, an increase in the level of fusions is indicative of the presence of cancer in the subject.

Methods of the invention include conducting a first, or initial assay to detect passenger fusions in DNA obtained from a sample obtained from a subject at a first point in time, and conducting a second, or subsequent assay at a second, or subsequent, point in time to determine persistence of the passenger fusions identified in the first sample, in a second sample obtained from the same subject. The methods of the invention may include quantifying the passenger fusions. For example, the passenger fusions may be detected by comparing nucleic acid obtained from a first sample to a reference germline sequence obtained from the same subject. Alternatively, the passenger fusions may be detected by comparing the DNA obtained from the sample obtained at the first point in time to germline DNA from the same subject. Comparing may include comparing a sequence of the DNA obtained from the sample obtained at the first point in time to the germline DNA of the same subject.

Methods of the invention may also include determining an increased amount of the fusions detected. For example, the subsequent, or second, assay may also include determining whether that sample contains an increased amount of the fusions detected in the first or initial sample. Determining if there is an increased amount of the fusions may include determining a rate of change of the passenger fusions over time. The passenger fusions may be quantified to determine a number of passenger fusions in the sample. An increased rate of change may be indicative of the presence of cancer in the subject.

Methods of the invention include enriching samples for nucleic acid containing passenger fusions in order to identify fusions that may be present in the sample at low abundance. For example, conducing a first assay to detect passenger fusions in DNA obtained from a sample obtained from a subject at a first point in time may include sequencing the DNA to detect the passenger fusions in the sample. Nucleic acid of a sample from the same subject may be enriched by introducing one or more Cas endonuclease/guide RNA complexes into the sample. The one or more guide RNAs target the passenger fusion in a sequence specific manner. The enrichment may be negative enrichment, whereby the method also includes introducing an exonuclease to the sample to digest nucleic acid to which the Cas ribonucleoprotein (RNP) are not bound. Methods for negative enrichment, including negative enrichment are taught, for example, in U.S. Pat. Nos. 10,370,700 and 10,081,829, the contents of each of which are incorporated herein by reference.

In addition, methods of the invention include amplifying fusions using primers that bind to regions in nucleic acids that flank a fusion. For example, one primer may be complementary to a sequence in nucleic acid on one side of the fusion, and the other primer may be complementary to a sequence in the nucleic acid on the other side of the fusion. The fusion may be amplified by the polymerase chain reaction (PCR).

Methods of the invention are useful to monitor cancer risk, regardless of the type or stage of the cancer. For example, the cancer may be breast cancer, colon cancer, gastric cancer, glioblastoma, leukemia, liposarcoma, liver cancer, lung cancer, lymphoma, medullablastoma, melanoma, oligoastrocytoma, oligodendroglioma, ovarian cancer, pancreatic cancer, prostate cancer, sarcoma, or thyroid cancer.

Methods of the invention may include providing a report containing diagnostic information and possible therapeutic interventions. For example, the report may contain information on one or more of the following: identify the passenger fusion; identify a cancer associated with the passenger fusion; prognosis for course of disease in the subject; probability that the disease or a particular clinical manifestation of the disease will return for the subject; likelihood that subject will develop resistance to a therapy or therapeutic agent; and a suggested course of the therapy for the subject.

The fusion may be detectable in a sample obtained from the subject prior to treatment of the subject for cancer. The fusion may be detectable in a sample obtained from the subject following treatment of the subject for cancer.

The fusion may have been previously detected in one or more samples obtained from another subject or group of subjects. Alternatively, the fusion may not have been previously detected in a sample obtained from another subject or group of subjects. The fusion may be associated with cancer in the same subject. The fusion may be associated with cancer in another subject or group of subjects.

### DETAILED DESCRIPTION

Methods of the invention recognize that the accumulation of fusions in genomic DNA over time, as opposed to the mere presence of such fusions, presents a sensitive diagnostic for cancer. For purposes of the invention, fusions resulting from deletions in either drivers or passengers may be used. However, it is the accumulation over time of passenger fusions that is often a leading indicator of disease. Specifically, the invention recognizes that it is insufficient to detect the presence of fusion mutations in somatic cell DNA. Rather, the informative diagnostic comes from the identification of rising levels of such mutations over time. While fusions are a hallmark of cancer, the mere presence of such mutations does not provide a reliable diagnostic indicator. The invention recognizes, however, that a detection of increasing levels of fusions provides a sensitive assay for cancer. In that regard, fusions, and in particular passenger fusions, are, according to the invention, a reliable biomarker for cancer. In this regard, fusions are analogous to prostate-specific antigen (PSA) as a biomarker for prostate cancer.

Methods of the invention also allow detection of fusions in a biological sample even when they are present in low abundance. Methods of the invention allow samples to be assayed in an inexpensive, quick, and reliable manner and thus are conducive to high throughput screening. In addition, methods described herein provide profiles of fusion specific to an individual patient. Therefore, they permit monitoring of cancer occurrence, or even recurrence, in an individual by detecting changes in an individual's profile of gene fusions over time.

Mutations that Contain or are Derived from Fusions

Somatic mutations that arise due to the genetic instability of cancer cells fall into two functional categories. The first functional category includes mutations that confer a selective growth advantage drive tumorigenesis; and thus are called “driver” mutations. Even though a driver mutation promotes uncontrolled growth of a cancer cell, a single driver mutation is usually not sufficient to convert a normal cell into a tumor cell. On the contrary, most cancer cells harbor from two to eight driver mutations. Vogelstein, et al., Cancer Genome Landscapes, Science. 2013 Mar. 29; 339(6127):1546-58, doi: 10.1126/science.1235122, the contents of which are incorporated herein by reference. The second category of functional mutations includes those that do not confer a selective growth advantage. Such mutations are called “passenger” mutations. It is estimated that 97% of mutations in cancer are passengers. Lawrence M S, et al., Discovery and saturation analysis of cancer genes across tumor types, Nature, 2014 Jan. 23; 505(7484):495-501. doi: 10.1038/nature12912, the contents of which are incorporated herein by reference. Driver mutations and passenger mutations are known in the art and described in more detail in, for example, Vogelstein, et al., Cancer Genome Landscapes, Science. 2013 Mar. 29; 339(6127):1546-58, doi: 10.1126/science.1235122; Lawrence M S, et al., Discovery and saturation analysis of cancer genes across tumor types, Nature, 2014 Jan 23; 505(7484):495-501. doi: 10.1038/nature12912; McFarland C D, et al., Cancer Res. 2017 Sep. 15; 77(18):4763-4772, doi: 10.1158/0008-5472.CAN-15-3283-T; and Pon, J R, and Marra, M A, Driver and passenger mutations in cancer, Annu Rev Pathol. 2015; 10:25-50. doi: 10.1146/annurev-pathol-012414-040312, the contents of each of which are incorporated herein by reference.

Somatic cell mutations in cancer cells also fall into two broad structural categories. In the first structural category are subtle somatic mutations that include small structural changes to DNA, such as single base substitutions and insertions or deletions of one or a few bases. Making up the second structural category are larger changes in chromosome structure, such as gene amplifications, deletions, inversions, and translocations. Such chromosomal rearrangements include fusions of DNA fragments or entire genes that are not contiguous in wild-type or unaltered chromosomal DNA. Fusions are useful as targets for analysis by polymerase chain reaction (PCR) because primers that bind to targets on opposite sides of the fusion site typically only produce a product if the rearrangement has occurred.

Methods of the invention include analysis of nucleic acids that contain fusions. A fusion may itself be a mutation, or may result in a mutation. For example and without limitation, the resultant mutation may contain or result from an amplification, deletion, duplication, insertion, inversion, or translocation.

A fusion may be characteristic of a particular type of cancer. For example, the fusion may have previously been detected in one or more reference subjects that have been diagnosed with a particular type of cancer.

A fusion or collection of fusions may be distinctive for an individual. For example, a fusion or a collection of fusions may have been detected in an initial sample obtained from an individual, thus forming a reference signature for the subject. The reference signature may or may not include a fusion or a collection of fusions previously associated with any type of cancer. The persistence or increased amount of the fusions, particularly passenger fusions, identified in the initial sample, in a test sample obtained from the same individual from a second point in time, or subsequent points in time, is indicative the presence of cancer in the individual. The fusions may be a signature for a particular type of tumor or cancer in the subject, i.e., they may be present in cancer cells or pre-cancerous cells of the test subject but not in normal cells of the subject.

Alternatively, a fusion or a collection of fusions differing from those detected in an initial sample may be detected in a test sample of an individual collected at a later point in time than the initial sample. The presence of newly identified fusion or collection of fusions may be a signature for a particular type of tumor or cancer in the subject, i.e., they may be present in cancer cells or pre-cancerous cells of the test subject but not in normal cells of the test subject.

### Sequencing

Methods of the invention include conducting a first assay to detect passenger fusions in DNA obtained from a sample obtained from a subject at a first point in time. Methods of the invention include sequencing DNA obtained from an initial sample obtained from the subject to obtain a sequence of the DNA, or initial sequence. The initial sequence may be the subject's whole genome, or large portions thereof. The sequence of DNA may be compared to a germline sequence obtained from the same subject. Comparing the sequence of DNA to the germline sequence allows for the detection of the presence of passenger fusions in the subject.

Sequencing may be by any method known in the art. See, generally, Quail, et al., 2012, A tale of three next generation sequencing platforms: comparison of Ion Torrent, Pacific Biosciences and Illumina MiSeq sequencers, BMC Genomics 13:341. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing.

Another example of a DNA sequencing technique that can be used is SOLiD technology by Applied Biosystems from Life Technologies Corporation (Carlsbad, Calif.). In SOLiD sequencing, genomic DNA is sheared into fragments, and adaptors are attached to generate a fragment library. Clonal bead populations are prepared in microreactors containing beads, primers, template, and PCR components. Following PCR, the templates are denatured and enriched and the sequence is determined by a process that includes sequential hybridization and ligation of fluorescently labeled oligonucleotides.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, each incorporated by reference. DNA is fragmented and given amplification and sequencing adapter oligos. The fragments can be attached to a surface. Addition of one or more nucleotides releases a proton (H+), which signal is detected and recorded in a sequencing instrument.

Another example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Other examples of a sequencing technology that can be used include the single molecule, real-time (SMRT) technology of Pacific Biosciences (Menlo Park, Calif.) and nanopore sequencing as described in Soni and Meller, 2007 Clin Chem 53:1996-2001. Such sequencing methods are useful when obtaining large fragments of DNA from a reference or test sample, such as in the methods described in U.S. Pub. 2018/0355408, the contents of which are incorporated by reference herein.

As described above, a fusion or a collection of fusions may be detected in the initial sample of the subject, thus forming a fusion reference signature for the subject. The reference signature may or may not include a fusion or a collection of fusions previously associated with any type of cancer. The germline sequence obtained from the same subject may act as the reference to which the sequence of the DNA obtained from a subject at a first point in time is compared. Detecting passenger fusions in a subject includes comparing a germline sequence of the subject to a sequence of DNA obtained from a sample obtained from a subject at first point in time. The germline sequence is obtained from a reference sample obtained from the same subject. The germline sequence may also be obtained by any of the above sequencing techniques. The germline sequence can be obtained from a buccal cell obtained via a buccal swab obtained from the subject and the test samples may be blood samples or tissue samples obtained from the same subject.

### Amplification of Nucleic Acids

Methods of the invention may optionally include amplifying nucleic acids that contain fusions. In some embodiments, one or more nucleic acids containing fusions are amplified by PCR using primers that bind to sequences flanking the fusion site, as described above. Any suitable type of PCR may be used. For example and without limitation, the PCR may be asymmetric PCR, hot-start PCR, ligation-mediated PCR, methylation-specific PCR (MSP), multiplex PCR, nested PCR, quantitative PCR, quantitative real-time PCR (QRT-PCR), reverse transcription PCR (RT-PCR), suicide PCR, or touchdown PCR. PCR methods are known in the art and described in, for example, Green, M R and Sambrook, J, eds., Molecular Cloning, A Laboratory Manual, Fourth Edition, Cold Spring Harbor Laboratory Press 2012, ISBN-13: 978-1936113415; Van Pelt-Verkuil, E., et al., Principles and Technical Aspects of PCR Amplification, Springer; 2008, ISBN-13: 978-1402062407; and Carl W. Dieffenbach and Gabriela S. Dveksler, eds., PCR Primer: A Laboratory Manual, Cold Spring Harbor Laboratory Press 2003, ISBN-13: 978-0879696542, the contents of each of which are incorporated herein by reference. Specifically, QRT-PCR may be used to quantify the fusions at any time point.

The nucleic acid may be DNA or RNA. The nucleic acid may by a subpopulation of DNA or RNA. For example and without limitation, the DNA may be cell-free DNA (cfDNA), circulating tumor DNA (ctDNA), or circulating cell-free mitochondrial DNA (ccf mtDNA). For example and without limitation, the RNA may mRNA, tRNA, rRNA, or snRNA.

The one or more amplified nucleic acids may have been previously detected in the subject, or they may not have been previously detected in the subject. Identification of one or more amplified nucleic acids not previously detected in the subject (i.e., not present in the reference sample of the subject) may be indicative of the presence of cancer. The one or more amplified nucleic acids may be detectable in a sample obtained from the subject prior to treatment of the subject for cancer, or they may not be detectable in a sample obtained from the subject prior to treatment of the subject for cancer. The one or more amplified nucleic acids may be detectable in a sample obtained from the subject following treatment of the subject for cancer, or they may not be detectable in a sample obtained from the subject following treatment of the subject for cancer. Identification of one or more amplified nucleic acids previously detected in a the subject (i.e., present in a test sample of the subject) may be indicative of failure of treatment or of the subject's resistance to treatment.

The methods may include amplification of multiple nucleic acids in a single reaction or assay. For example, the method may include amplifying 2, 3, 4, 5, 6, 7, 8, 9, 10, 11, 12, 13, 14, 15, 16, 17, 18, 19, 20, about 25, about 30, about 40, about 50, or more nucleic acids in a single reaction or assay. Each nucleic acid may contain a fusion or a mutation containing or resulting from a fusion. Each nucleic acid may contain a different mutation or fusion. Alternatively or additionally, two or more of the nucleic acids may contain the fusion or mutation, i.e., two or more of the nucleic acids may overlap.

### Detection of Fusions

Detection of fusions can be accomplished by methods of the art. For example and without limitation, detection may include chromatography, DNA staining, electron microscopy, electrophoresis, fluorescence (e.g., fluorescence imaging, fluorescence microscopy, fluorescent probe hybridization, or fluorescence resonance energy transfer), immunomagnetic separation, optical microscopy, sequencing, spectrophotometry, or combinations thereof. Methods of detecting nucleic acids are known in the art and described in, for example, Green, M R and Sambrook, J, eds., Molecular Cloning, A Laboratory Manual, Fourth Edition, Cold Spring Harbor Laboratory Press 2012, ISBN-13: 978-1936113415; Van Pelt-Verkuil, E., et al., Principles and Technical Aspects of PCR Amplification, Springer; 2008, ISBN-13: 978-1402062407; and Carl W. Dieffenbach and Gabriela S. Dveksler, eds., PCR Primer: A Laboratory Manual, Cold Spring Harbor Laboratory Press 2003, ISBN-13: 978-0879696542; Peterson, 2009, Generations of sequencing technologies, Genomics 93(2):105-11; Goodwin, 2016, Coming of age: ten years of next-generation sequencing technologies, Nat Rev Genet 17(6):333-51; Morey, 2013, A glimpse into past, present, and future DNA sequencing, Mol Genet Metab 110(1-2):3-24; Xu, 2014, Label-Free DNA Sequence Detection through FRET from a Fluorescent Polymer with Pyrene Excimer to SG, ACS Macro Lett 3(9):845-848; Safarik and Safarikova, Magnetic techniques for the isolation and purification of proteins and peptides, Biomagn Res Technol. 2004; 2:7, doi: 10.1186/1477-044X-2-7; Ballou, David P.; Benore, Marilee; Ninfa, Alexander J. (2008) Fundamental laboratory approaches for biochemistry and biotechnology (2nd ed.), Hoboken, N.J.: Wiley, p. 129. ISBN 9780470087664; Striegel, A. M. et al., Modern Size Exclusion Chromatography, Practice of Gel Permeation and Gel Filtration Chromatography, 2nd ed., Wiley: NY, 2009; Small, Hamish (1989), Ion chromatography, New York: Plenum Press, ISBN 0-306-43290-0; Tatjana Weiss, and Joachim Weiss (2005), Handbook of Ion Chromatography, Weinheim: Wiley-VCH, ISBN 3-527-28701-9; Gjerde, Douglas T. and Fritz, James S. (2000), Ion Chromatography, Weinheim: Wiley-VCH, ISBN 3-527-29914-9; Jackson and Haddad (1990), Ion chromatography: principles and applications, Amsterdam: Elsevier, ISBN 0-444-88232-4, Cady, 2003, Nucleic acid purification using microfabricated silicon structures, Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191;

Wolfe, 2002, Toward a microchip-based solid-phase extraction method for isolation of nucleic acids, Electrophoresis 23:727-733; and U.S. Pat. No. 8,318,445, the contents of each of which are incorporated herein by reference.

### Samples

Methods of the invention require the collection of different samples from the same subject over a period of time. Methods of the invention include obtaining samples containing germline DNA. Methods of the invention include obtaining samples containing passenger fusions.

For example and without limitation, a sample may be or include one or more of bile, blood, bone marrow, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, and vomit. The sample may be a tissue sample from an animal. The tissue sample may be from the skin, conjunctiva, gastrointestinal tract, respiratory tract, vagina, placenta, uterus, oral cavity or nasal cavity.

The sample may be obtained by any method. For example and without limitation, the sample may be obtained by aspiration with a needle, liquid biopsy, tissue biopsy, buccal swab, nasal swab, mouthwash, or spit kit.

In one embodiment, patient samples are obtained from both germline sources (e.g., buccal cells) and from blood or tumor tissue. For example, a sample obtained from a subject at a first point in time (initial sample) may be any sample that contains DNA. The sample may be blood or tumor tissue. The DNA may be compared to a germline sequence obtained from the same subject. The germline DNA may be obtained from any type of biological sample of the subject that contains germline DNA. Preferably, the germline DNA is obtained from buccal cells. The initial sample and subsequent samples may be obtained from a suspected tumor or from blood, including plasma and serum.

In embodiments of the invention, an initial sample or reference sample is obtained from a subject and subsequent test samples are obtained from the subject at later time points. For example, nucleic acid obtain in a reference sample may be compared to a reference germline sequence from the same subject to detect fusions. The detected fusions may be detected in a second or subsequent sample obtained from the same subject. The germline sequence may be obtained from a buccal cell of the subject. The nucleic acid of the reference/initial sample and the subsequent samples may be obtained from blood or tissue samples obtained from the same subject.

When multiple test samples are used, the method may include multiple amplification steps. Thus, the methods may include obtaining multiple test samples and independently amplifying nucleic acids from the samples independently. For example, different test samples from different subjects may be processed sequentially. For example, a series of test samples may be obtained from a subject at different times, and each test sample may be processed after it is obtained and before the next test sample is obtained.

### Diseases

Methods of the invention are particularly useful for detecting disease in subjects with no prior history of disease. The disease may be cancer. The cancer may be any type of cancer. The subject may or may not have clinical signs of any type of cancer or disease. The cancer may be previously associated with a particular gene fusion or collection of gene fusions, or the cancer may have no previous association with any gene fusions. For example and without limitation, the cancer may be breast cancer, colon cancer, gastric cancer, glioblastoma, leukemia, liposarcoma, liver cancer, lung cancer, lymphoma, medullablastoma, melanoma, oligoastrocytoma, oligodendroglioma, ovarian cancer, pancreatic cancer, prostate cancer, sarcoma, or thyroid cancer.

Accordingly, the invention provides methods of detecting cancer by monitoring the accumulation of passenger fusions over time. Thus, methods of the invention include using passenger fusions as an analyte in an assay to detect cancer. In methods of the invention, passenger fusions may be assessed in bulk to determine generally whether the quantity of mutations is increasing or may themselves be quantified, preferably on a weighted basis with respect to a standard, over time. In one embodiment patient samples are obtained from both germline sources (e.g., buccal cells) and from blood or tumor tissue. Passenger mutations are identified by comparing DNA obtained from blood or tumor tissue to germline DNA. If fusions in passenger genes (passenger mutations) are detected, the analysis is repeated at future time points in order to determine a rate of change of the passenger fusions. The rate of change may be quantitative or based on bulk measurement. For example the slope of a curve defining a rate of passenger fusion accumulation defines the acceleration of passenger fusion accumulation, which is then assessed to provide an informative diagnosis.

Methods of the invention include quantifying passenger fusions in a sample obtained from a subject at an initial time, or first point in time, and at a subsequent time, wherein an increase in the number or amount of such mutations over time is indicative of cancer. For example, a passenger fusion may be identified in a subject by comparing a whole-genome sequence of germline DNA of the subject to DNA obtained from blood or tissue to identify passenger fusions in the latter. The germline DNA may be obtained from any type of biological sample of the subject that contains germline DNA. Preferably, the germline DNA is obtained from buccal cells. The test DNA may be obtained from a suspected tumor or from blood, including plasma and serum. Any passenger fusions identified in test samples are specific to the subject, and thus are a patient-specific biomarker that is used to monitor the subject.

Methods of the invention include obtaining a baseline level of the passenger fusion in an initial test sample obtained from the subject. The baseline level, or the initial passenger fusion index, may be obtained by quantifying any passenger fusions identified in the sample by comparison to the germline DNA of the same patient. The initial passenger fusion index may be used to compare to the levels of the passenger fusion in subsequent samples obtained from the same patient.

Methods of the invention include quantifying passenger fusions in a subsequent sample obtained from the subject at a later time to determine whether there is an increase in the level of the passenger fusions. Subsequent samples may be obtained at any future time point from the subject. For example, a subsequent sample may be obtained monthly, yearly, or at any frequency. Identifying an increase in the level of passenger mutations in subsequent samples may indicate the presence of cancer in the subject. The frequency of obtaining subsequent samples may be increased if an increase in the level of the passenger fusion is identified at any time point. For example, subsequent samples may be obtained on a monthly basis to monitor the increasing level of the passenger fusions. The frequency of the subsequent samples may be decreased if a decrease in the level of the passenger fusion is identified. Each time, comparing the level of passenger fusions in the subsequent sample to the baseline level, thereby monitoring for an increase in the level of passenger fusions. Alternatively, the level of passenger fusions of the subsequent sample may be compared to a previous sample. Regardless, an increase in the level of fusions is indicative of the presence of cancer in the subject.

Methods of the invention include conducting a first, or initial assay to detect passenger fusions in DNA obtained from a sample obtained from a subject at a first point in time, and conducting a second, or subsequent assay at a second, or subsequent, point in time to determine persistence of the passenger fusions identified in the first sample, in a second sample obtained from the same subject. In an embodiment, the passenger fusions may be detected by comparing nucleic acid obtained from a first sample to a reference germline sequence obtained from the same subject. The passenger fusions may be detected by comparing the DNA obtained from the sample obtained at the first point in time to germline DNA from the same subject. Comparing a sequence of the DNA obtained from the sample obtained at the first point in time to the germline DNA of the same subject allows for the identification of passenger fusions specific to the subject. Monitoring the rate of change of the fusions in samples obtained at later point in time provides a means for detecting disease in the subject. Detecting an increased amount of the fusions in samples obtained at a second point in time or over time is indicative of disease in the subject. Particularly, detecting an increased amount of passenger fusions over time is indicative of cancer in the subject.

Methods of the invention may also be useful for detecting residual disease in subjects that have already received treatment for the disease. The utility of the methods does not depend on the nature of prior treatment. Thus, the prior treatment may be treatment by any method for any duration. For example, in embodiments in which the disease is cancer, the prior treatment may be or include one or more of angiogenesis inhibitor therapy, chemotherapy, hormonal therapy, immunotherapy, radiation therapy, surgery, or a targeted therapy, e.g., monoclonal antibody therapy, peptide therapy, photodynamic therapy, or ultrasound therapy. The prior treatment may have been completed, or it may be ongoing. The disease may be active, in partial remission, or in complete remission. The disease may have been in remission, e.g., partial remission or complete remission, in the subject for a defined period prior to obtaining a reference sample for analysis. For example, the disease may have been in remission in the subject for about 3 months, about 6 months, about 9 months, about 1 year, about 2 years, about 3 years, about 4 years, about 5 years, about 6 years, about 8 years, about 10 years, about 15 years, about 20 years, or more. Identification of persisting gene fusions or newly identified gene fusions in later obtained test samples from the subject may be indicative of ineffective treatment or relapse of the disease.

### Reports

In certain embodiments, the methods include providing a report containing information about the cancer in the subject based on analysis of the fusions. The report may contain information on one or more of the following: identifying the fusion in the test sample, identify a type of cancer associated with the fusion, prognosis for course of disease in the subject, probability that the disease or a particular clinical manifestation of the disease will return for the subject, likelihood that the subject will develop resistance to a therapy or therapeutic agent, and a suggested course of the therapy for the subject. For example and without limitation, the element of a course of therapy may be or include type of therapy, therapeutic agent, drug dosage, frequency of administration, duration. In other embodiments, the report may also contain information such as: estimate of the subject's tumor mutational burden; efficacy of the subject's prior treatment; development of resistance to a therapy or therapeutic agent in the subject's prior treatment; and likelihood that subject will develop resistance to a subsequent therapy or therapeutic agent. If the subject had prior treatment, the suggested course of the therapy may have one or more elements that are different from the subject's prior treatment.

Enrichment of Nucleic Acids that Contain or Result from Fusions

Methods of the invention may include enriching the test sample for nucleic acids that contain fusions. The enrichment may be performed prior to amplification to facilitate detection of very rare cancer cells, which may not otherwise be detectable. Alternatively or additionally, the enrichment may be performed subsequent to amplification as a secondary analytical step to confirm results from the amplification analysis and/or to obtain further information about the fusions present in a sample.

Nucleic acids containing fusions may be enriched by using programmable nuclease, such as a CRISPR-associated (Cas) endonuclease, zinc-finger nuclease (ZFN), transcription activator-like effector nuclease (TALEN), or RNA-guided engineered nuclease (RGEN). Programmable nucleases can be engineered to bind to specific DNA sequences. Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

One approach involve the use of a programmable nuclease to enrich a sample for passenger fusions is to target a programmable nucleases to sequences on each side of a fusion site and then use an exonuclease to digest DNA that is not bound by the programmable nuclease. Most DNA sequences are degraded by the exonuclease, but the fusion site, which is flanked by the bound programmable nuclease, is protected from degradation. Another approach is to target a programmable nuclease to bind a sequence that comprises that fusion site and then use an exonuclease to digest DNA that is not bound by the programmable nuclease. Here again, the fusion site is protected from degradation by binding of the programmable nuclease. These approaches are described in detail in, for example, International Patent Publication Nos. WO 2018/231945; WO 2018/231946; and WO 2018/231963, the contents of each of which are incorporated herein by reference.

Programmable nucleases are generally able to cleave DNA at or near the sites to which they bind. Cleavage of the target nucleic acid may inhibit detection. Therefore, in certain embodiments, the programmable is enzymatically inactive. The use of enzymatically inactive programmable nucleases is described in, for example, International Patent Publication Nos. WO 2018/231945; WO 2018/231946; and WO 2018/231963, the contents of each of which are incorporated herein by reference.

Any suitable exonuclease may be used to digest unprotected nucleic acids. For example, the exonuclease may be Lambda exonuclease, RecJf, Exonuclease III, Exonuclease I, Exonuclease T, Exonuclease V, Exonuclease VII, T5 Exonuclease, or T7 Exonuclease. Combinations of exonucleases may be used.

Another approach to enrich for fusions is to amplify nucleic acids using modified nucleotides and then use an exonuclease to digest unmodified nucleic acids. Modified nucleic acids, such as DNA having nucleotides joined by phosphorothioate linkages, are not substrates for exonucleases and thus are protected from degradation. One or more of the aforementioned exonucleases may be used to digest unmodified nucleic acids. The use of modified nucleotides to enrich for nucleic acids is described in, for example, International Patent Publication Nos. WO 2018/231955; WO 2018/231967; and WO 2018/231985, the contents of each of which are incorporated herein by reference.

Nucleic acids enriched by the foregoing processes may be further enriched by purification. For example, nucleic acids may be purified by chromatography, electrophoresis, immunomagnetic purification.

Following enrichment of the nucleic acid that contains or results from a gene fusion, the nucleic acid may be detected. Any of the detection methods described above may be used.

### Kits

The invention also provides kits for performing the methods of the invention. The kit may include any reagent or material useful for performing the methods. For example, the kit may include primers that bind to sequences that flank fusions. The kit may include reagents for enrichment steps, such as guide RNAs for use with Cas endonucleases and nucleic acids, such as DNA or mRNA, that encode a Cas endonuclease. The kit may include materials for sample preparation. The kit may include instructional materials for performing the methods.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of detecting for cancer in a subject, the method comprising:
conducting a first assay to detect passenger fusions in DNA obtained from a sample obtained from a subject at a first point in time; and
conducting a second assay at a second point in time to determine persistence of said passenger fusions in a second sample obtained from said subject.

2. The method of claim 1, wherein said passenger fusions are detected by comparison of nucleic acid obtained in said sample to a reference germline sequence obtained from said subject.

3. The method of claim 1, wherein said fusions are quantified.

4. The method of claim 1, wherein said second assay further comprises determining whether said second sample contains an increased amount of said passenger fusions.

5. The method of claim 4, wherein said determining step comprises determining a rate of change of said passenger fusions.

6. The method of claim 1, wherein said passenger fusions are identified by comparing said DNA to germline DNA from the subject.

7. The method of claim 6, wherein said comparing comprises comparing a sequence of said DNA to said germline DNA.

8. The method of claim 1, wherein said first assay comprises sequencing DNA obtained from said sample.

9. The method of claim 7, further comprising enriching the second sample for identified passenger fusions.

10. The method of claim 9, wherein the enriching step comprises introducing one or more Cas endonuclease/guide RNA complexes into the second sample wherein guide RNAs target the identified passenger fusion in a sequence-specific manner.

11. The method of claim 10, further comprising introducing an exonuclease to digest nucleic acid to which said complexes are not bound.

12. The method of claim 1, further comprising providing a diagnostic report.
